(19) United States
(12) Patent Application Publication
(10) Pub. No.: US 2012/0288549 A1
(43) Pub. Date: Nov. 15, 2012

(54) COSMETIC COMPOSITION CONTAINING WHITE GINSENG POWDER

(76) Inventors: SunSang Kwon, Yongin-si (KR); Go Un Han, Seoul (KR); Eun Joo Kim, Suwon-si Gyeonggi-do (KR); Duck Hee Kim, Seoul (KR); Han-Kon Kim, Suwon-si Gyeonggi-do (KR)

(21) Appl. No.: 13/557,733

(22) Filed: Jul. 25, 2012

Related U.S. Application Data
(63) Continuation of application No. 13/264,637, filed on Oct. 14, 2011.

(30) Foreign Application Priority Data
Apr. 21, 2009 (KR) 10-2009-0034794

(51) Int. Cl. A61K 36/258; A61K 8/02; A61P 17/00; A61Q 1/12; A61K 9/14; A61K 8/97

(52) U.S. Cl. 424/401; 424/728; 424/400; 424/69

(57) ABSTRACT

The present invention relates to a cosmetic composition containing white ginseng powder. Applying a cosmetics containing white ginseng powder to the skin of a user stimulates blood circulation and exhibits superior skin scrubbing effect.

### TECHNICAL FIELD

The present disclosure relates to a cosmetic composition.

### BACKGROUND ART

Ginseng (Panax ginseng C. A. Meyer) is a plant belonging to the genus Panax of the family Araliaceae. It has been used in Korea, China, Japan and other regions as herbal medicine for more than 2,000 years, in order to prevent diseases and extend life span. Currently known functions and effects of ginseng include anticancer activity, immunomodulatory activity, antidiabetic activity, liver function promoting activity, cardiovascular disorder improving activity, anti-arteriosclerotic activity, blood pressure controlling activity, menopausal disorder improving activity, anti-stress and anti-fatigue activity, antioxidant activity, anti-aging activity, and the like (Recent Korean Ginseng ‘Constituents and Effects’, Korea Ginseng and Tobacco Research Institute, 56-112, 1996).

Also, since ginseng is known to restore the skin damaged by aging and oxidative stress, provide sufficient nutrients and oxygen to the skin by stimulating blood circulation in the skin, and provide good moisturizing effect, it has been used in cosmetics. In particular, it is usual to dry ginseng extract and make it into powder for use.

### DISCLOSURE

### Technical Problem

The present disclosure is directed to providing a sustained-release cosmetic composition containing the natural white ginseng as it is and providing good skin scrubbing effect and stimulating blood circulation in the skin.

### Technical Solution

In one general aspect, the present disclosure provides a cosmetic composition containing white ginseng powder obtained by grinding white ginseng. In another general aspect, the present disclosure provides a cosmetic composition containing white ginseng powder having a particle size of 150-450 μm.

### Advantageous Effects

The cosmetic composition containing white ginseng powder according to the present disclosure contains powder obtained by grinding natural white ginseng as it is. Since it contains cellulose, it provides scrubbing effect. Further, the inherent fragrance of ginseng is maintained better than the existing extracts, and the composition can provide invigorating effect by stimulating blood circulation in the skin. In addition, the composition can provide long-lasting sustained release effect.

### DESCRIPTION OF DRAWINGS

FIG. 1 shows change in blood circulation on the face before and after applying a formulation containing 5 wt % of white ginseng powder or a formulation not containing white ginseng powder once or 5 times on the face;

FIG. 2 shows IR images obtained before and after applying a formulation containing 5 wt % of white ginseng powder or a formulation not containing white ginseng powder once or 5 times on the face; and

FIG. 3 shows change in the amount of dead skin cells on the face before and after applying a formulation containing 5 wt % of white ginseng powder or a formulation not containing white ginseng powder once or 5 times on the face.

### BEST MODE

In an aspect, the present disclosure provides a cosmetic composition comprising white ginseng powder obtained by grinding white ginseng as an active ingredient.

The inventors of the present disclosure have studied to broaden the applicability of ginseng extract to the skin. As a result, they have found out that, when white ginseng, which can be said as the gist of the ginseng extract, is introduced into cosmetics as it is, it can improve skin scrubbing effect and stimulate blood circulation in the skin. Also, they have found out that, when white ginseng powder obtained by removing the outer skin of ginseng is introduced into cosmetics, the active ingredient of ginseng is released slowly in a sustained manner. In addition, a cosmetic composition according to an embodiment of the present disclosure can solve the safety and stability problems associated with release of the conventional compositions with the active ingredient artificially encapsulated in polymer microcapsules or nanocapsules.

As used herein, “white ginseng” refers to a ginseng obtained by drying fresh ginseng, after slightly peeling the skin without steaming, so that the water content does not exceed 14%. It has pale yellow to white color. Since white ginseng less than 2 years old has slightly less active ingredient content and one older than 7 years is costly to grow, one which is 3-6 years old may be used. Most specifically, 4-year-old white ginseng may be used.

In one aspect of the present disclosure, the white ginseng powder may have a particle size of 150-450 μm. When the particle size is larger, the user may feel unpleasant due to foreign body sensation. Also, when the particle size is smaller, the scrubbing effect may be insufficient.

In one aspect of the present disclosure, the white ginseng powder may be included in an amount of 0.1-20 wt % based on the total weight of the composition. When the content is below 0.1 wt %, the effect intended by the present disclosure may be only slight. Also, when the content is more than 20 wt %, the stability of the composition may decrease.

In one aspect, the present disclosure provides a cosmetic composition comprising white ginseng powder as an active ingredient. The cosmetic composition may comprise a cosmetically or dermatologically acceptable medium or base. It may be prepared into any topically applicable formulation, for example, solution, gel, solid, anhydrous paste, oil-in-water emulsion, water-in-oil emulsion, multiple emulsion, suspension, micro emulsion, microcapsule, ionic (liposome) or nonionic vesicular dispersion agent, foam or patch. The composition may be prepared according to a method commonly employed in the art.

The cosmetic composition may further comprise, in addition to the above-described substances, other ingredients that may provide synergism to the desired effect, without negatively affecting the desired effect. The other ingredients may be appropriately selected and added by those skilled in the art without difficulty depending on the particular formulation or purpose of the cosmetic composition.

For example, the cosmetic composition may comprise a transdermal absorption promoting substance. Also, the cosmetic composition of the present disclosure may comprise a substance selected from a group consisting of water-soluble vitamin, oil-soluble vitamin, polypeptide, polysaccharide, sphingolipid, and seaweed extract. In addition, the cosmetic composition of the present disclosure may comprise, along with the active ingredient, other ingredients commonly used in cosmetic compositions, if necessary. Examples include oil, fat, humectant, emollient, surfactant, organic or inorganic pigment, organic particle, UV absorbent, antiseptic, sterilizer, antioxidant, plant extract, pH control agent, alcohol, coloring agent, fragrance, blood circulation stimulant, cooling agent, antiperspirant, purified water, and the like. However, the other ingredients that may be included in the cosmetic composition are not limited thereto. Also, the amount of the other ingredients included in the cosmetic composition may be determined within the range without negatively affecting the purpose and effect of the present disclosure.

The formulation of the cosmetic composition is not particularly limited, but may be selected appropriately depending on desired purposes. For example, it may be formulated as toner, emulsion, essence, creams including cleansing cream, massage cream or nourishing cream, base makeup products including makeup base, powder, twin cake, foundation or BB cream, point makeup products for eye makeup or lip makeup, pack, or the like.

### MODE FOR INVENTION

The examples and experiments will now be described. The following examples and experiments are for illustrative purposes only and not intended to limit the scope of the present disclosure.

### Preparation Example 1

### Preparation of White Ginseng Powder

Dried 4-year-old white ginseng was ground. A pin mill having superior grinding effect was used and the rotation speed was maintained at 4000-6000 rpm. The ground white ginseng was passed through a 450-μm sieve (40 mesh). Then, the white ginseng powder that passed therethrough was passed again through a 150-μm sieve (100 mesh). The white ginseng powder having a particle size smaller than 150 μm passed through the 100-mesh sieve. The white ginseng powder that did not pass through the 100-mesh sieve was collected. Thus, the white ginseng powder of this example has a particle size of 150-450 μm.

### Comparative Preparation Example 1

The white ginseng powder that did not pass through the 450-μm sieve in Preparation Example 1 was collected.

### Comparative Preparation Example 2

The white ginseng powder that passed through the 150-μm in Preparation Example 1 was collected.

### Example 1

### Preparation of Formulation Containing White Ginseng Powder

The white ginseng powder prepared in Preparation Example 1 was mixed with other ingredients as described in Table 1.

### Comparative Example 1

The white ginseng powder prepared in Comparative Preparation Example 1 was mixed with other ingredients as described in Table 1.

### Comparative Example 2

The white ginseng powder prepared in Comparative Preparation Example 2 was mixed with other ingredients as described in Table 1.

TABLE 1

| | | Comparative | Comparative |
|---|---|---|---|
| | Example 1 | Example 1 | Example 2 |
| Ingredients | (wt %) | (wt %) | (wt %) |
| Disodium methylene | 0.02 | 0.02 | 0.02 |
| diamine tetraacetate | | | |
| Ethoxyglycol | 1.00 | 1.00 | 1.00 |
| Polyacrylate | 20.00 | 20.00 | 20.00 |
| Ethanol | 30.00 | 30.00 | 30.00 |
| White ginseng powder | 5.0 (Prep. | 5.0 (Comp. | 5.0 (Comp. |
| | Ex. 1) | Prep. Ex. 1) | Prep. Ex. 2) |
| Hydrogenated castor oil | 0.80 | 0.80 | 0.80 |
| Phenyltrimethicone | 0.20 | 0.20 | 0.20 |
| Triethanolamine | 0.40 | 0.40 | 0.40 |
| Aromatic | a little | a little | a little |
| Distilled water | To make 100 | To make 100 | To make 100 |

### Test Example 1

### Scrubbing Effect Depending on Particle Size of White Ginseng Powder

Scrubbing effect on the face was compared using the formulations containing 5 wt % of the white ginseng powder prepared in Example 1 and Comparative Examples 1-2

For twenty women in their 20s and 30s, sensory test was carried out on a 7-point scale. The result is shown in Table 2. In Table 2, the white ginseng powder of Example 1 has a particle size of 150-450 μm, the white ginseng powder of Comparative Example 1 has a particle size larger than 450 μm, and the white ginseng powder of Comparative Example 2 has a particle size smaller than 150 μm. After attaching packs on their faces, the subjects were asked to score their feeling with 1, 3, 5 or 7 points. The higher score translates into better feeling.

TABLE 2

| | Feeling after pack | |
|---|---|---|
| Test substances | attachment | Comments |
| Example 1 | 7 | — |
| Comparative Example 1 | 3 | Too rough and irritable |
| Comparative Example 2 | 1 | Scrubbing effect is |
| | | insignificant |

As seen from Table 2, the pack of Example 1 provided the best feeling. Thus, it can be seen that the white ginseng powder having a particle size of 150-450 μm is suitable to provide the scrubbing effect with no foreign body sensation.

### Test Example 2

### Blood Circulation Stimulating Effect

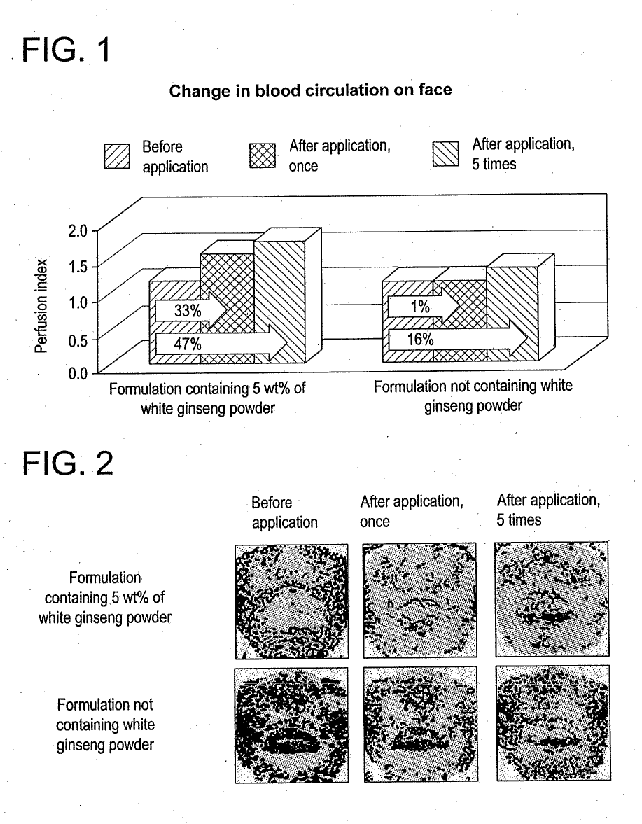

In order to test the blood circulation stimulating effect of the white ginseng powder, twenty subjects were divided into two groups. Test was performed with a formulation not containing white ginseng powder as control. As for the test group, the formulation of Example 1 was applied to the skin. FIG. 1 shows the change in blood circulation on the face before and after applying the formulation of the control group or the test group once or 5 times on the face. Also, the change in skin temperature was measured before and after applying the formulations once or 5 times using an IR camera. FIG. 2 shows the result.

As seen from FIG. 1 and FIG. 2, an increase of the blood circulation in the skin and skin temperature was higher over the entire face, including forehead, cheeks and chin, when the formulation containing the white ginseng powder was applied as compared to when the formulation not containing white ginseng powder was applied. In the IR images of FIG. 2, the brighter area is where the skin temperature is higher. It can be seen that the bright area is broader when the formulation containing the white ginseng powder was applied—particularly, when the formulation was applied 5 times, the skin temperature increased entirely—as compared to when the formulation not containing white ginseng powder was applied. That is to say, as a result of analyzing the change in skin temperature after using the formulation once or 5 times, the white ginseng powder group showed significant increase in skin temperature on the entire surface as compared to the control group. Therefore, it can be concluded that the formulation containing the white ginseng powder of the present disclosure stimulates blood circulation in the skin and thus leads to increase of skin temperature.

### Test Example 3

### Effect of Decreasing Dead Skin Cells on Face

In order to test the dead skin cells decreasing effect of the white ginseng powder on the face, twenty subjects were divided into two groups. Test was performed with a formulation not containing white ginseng powder as control. As for the test group, the formulation of Example 1 was applied to the skin.

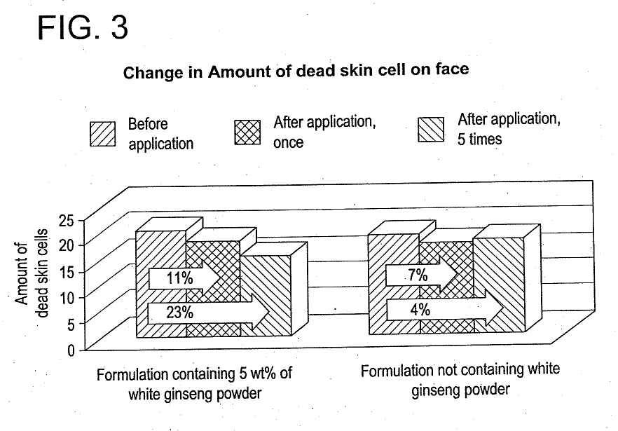

As seen from FIG. 3, the dead skin cell decreasing effect could be identified when the formulation containing the white ginseng powder was applied 5 times.

1-4. (canceled)

5. A method for stimulating blood circulation of a subject, comprising:
administering an effective amount of white ginseng powder to the subject, the white ginseng powder obtained by grinding white ginseng.

6. The method according to claim 5, wherein the white ginseng powder has a particle size of 150-450 μm.

7. The method according to claim 5, wherein the white ginseng is 3-6 years old.

8. The method according to claim 5, wherein the white ginseng powder is administered in the form of a cosmetic composition, and the cosmetic composition comprises the white ginseng powder in an amount of 0.1-20 wt % based on the total weight of the composition.

9. The method according to claim 5, wherein the white ginseng powder is transdermally administered to the subject.
